(19) United States
(12) Patent Application Publication
(10) Pub. No.: US 2022/0362571 A1
(43) Pub. Date: Nov. 17, 2022

(54) DEVICE COMPRISING A HEAT SOURCE

(71) Applicant: REMEDEE LABS, MEYLAN (FR)

(72) Inventors: Aurélien LAMBERT, FONTAINE (FR); Michael FOERSTER, CORENC (FR)

(21) Appl. No.: 17/434,932

(22) PCT Filed: Feb. 27, 2020
(86) PCT No.: PCT/EP2020/055078

§ 371 Date: Aug. 30, 2021

(30) Foreign Application Priority Data
Mar. 1, 2019 (FR) 1902167

(51) Int. Cl. A61N 5/02

(52) U.S. Cl. CPC A61N 5/025; A44C 5/0023

(57) ABSTRACT

The invention relates to a device (10) comprising at least one housing (2) having a heat source (1), and at least one strand (11, 12) forming a more flexible pad than the housing (2). The strand (11, 12) comprises two layers (14, 19) of heat-conducting material and a layer (18) of phase change material situated between the two layers of heat-conducting material.

### CROSS-REFERENCE TO RELATED APPLICATIONS

This patent application is a United States National Stage of International Patent Application No. PCT/EP2020/055078, filed on Feb. 27, 2020, which in turn claims the benefit of French Patent Application No. 1902167, filed on Mar. 1, 2019. The entire disclosures of the above patent applications are incorporated herein by reference.

### FIELD

The invention relates to devices containing electronics. It relates in particular to the management of heat dissipation in a device intended to treat pain by the transmission of millimeter waves.

### BACKGROUND

The state of the art, in particular in document U.S. Patent Application Publication No. (Delano), already describes a connected bracelet comprising a heat dissipation system including two heat-conducting layers surrounding a layer of phase change material. This stack dissipates some of the heat emitted by the electronics of the bracelet, but not sufficiently in case of high heat emission.

However, for the comfort of the human subject wearing the bracelet, the skin temperature must be maintained below a predetermined value.

### SUMMARY

The device of the present disclosure aims in particular to improve the management of heat dissipation in a connected device.

This device therefore relates to a device including:
at least one housing having a heat source, and
at least one strand forming a more flexible part than the housing, the strand including:
two layers of heat-conducting material, and
a layer of phase change material situated between the two layers of heat-conducting material.

Thus, the heat dissipation management takes advantage of the length of the strand, or handle or arc, of the device to maximize the area for dissipation of the heat emitted by the source, the heat dissipating along the entire length of one of the layers of heat-conducting material, then within the layer of phase change material, then, for the remainder, along the entire length of the other conducting layer. Dissipation is therefore not confined to the housing where the heat is emitted, but extends within a flexible part outside the housing. The maximum quantity of heat is therefore dissipated and the temperature of the device in contact with the skin, at the heat source, is reduced.

Advantageously, the device is a bracelet.

A “bracelet” means any ring-shaped object which can be worn, for example around the wrist, the arm or an ankle. The invention reduces the heat of the bracelet in contact with the skin, for example in contact with the wrist.

Preferably, the housing is rigid.

Thus, the device is used to manage the heat dissipation of the device while the heat source is not easily deformable or movable.

Advantageously, a thermal conductivity value of at least one of the heat-conducting layers is greater than or equal to 350 watts per meter-kelvin, preferably greater than or equal to 1300 watts per meter-kelvin.

The value of 350 watts per meter-kelvin corresponds approximately to the thermal conductivity of copper and tests have demonstrated the sufficiency of copper sheets as heat-conducting layers under some conditions. Note, however, that the greater the conductivity, the more the thickness of the layer can be reduced for a given dissipation level. Layers of conductivity greater than or equal to 1300 watts per meter-kelvin can thus be very thin, thereby saving space within the device.

Preferably, the thickness of at least one of the heat-conducting layers is between 0.01 and 1 millimeter.

Thus, the more conducting the layer, the thinner it can be. The device can therefore also be thin, which is comfortable, aesthetically interesting or cheaper to produce.

Advantageously, the phase change enthalpy of the layer of phase change material is approximately 100 joules per gram.

Generally, the higher the enthalpy, the more the phase change material will be able to absorb heat, which is interesting in particular in case of a transient state where the heat source supplies high thermal power over a short period.

Preferably, the two layers of heat-conducting material are bonded to two opposite surfaces of the layer of phase change material.

Thus, the three layers form a compact stack, the layers being possibly and uniquely separated by two adhesive coats situated between a first conducting layer and the layer of phase change material firstly, between, the latter and the second heat-conducting layer secondly. The heat is therefore transmitted directly from one layer of the stack to the other, which improves its dissipation while reducing the space occupied by the stack formed by the three layers.

Advantageously, at least one of the three layers also extends into the housing.

Thus, the or each layer is as close as possible to the heat source, in the housing which can in particular be in contact with the lower or upper surface of the wrist of a human subject. The or each layer also extends outside the housing, within the strand forming a flexible part and closing the device. The heat thus leaves from the rigid part, the housing where the heat source is situated, and is dissipated as it extends along the device.

Preferably, the device further including an electronic connection flex extending from the housing and into the strand, at least one of the layers of heat-conducting material being attached to the flex in the strand.

Thus, the connection flex can be used to connect the electronics of the housing with another element of the device. This other element may correspond to electronic elements situated outside the housing, even in another housing, for example situated opposite the first housing. Thus, the layer takes advantage of the presence of this flex to extend into the strand. It therefore uses the presence of an element which is already there for other purposes—connect the electronics—to extend into the strand, which avoids making the device structure more complex.

Advantageously, at least one of the layers of heat-conducting material comprises a sheet of pyrolytic graphite of thickness between 10 and 100 micrometers.

This sheet of graphite has in fact a high heat-conducting power, about five times greater than that of copper, while being thin. The final thickness depends on the required arrangement of the device.

Preferably, the thickness of the layer of phase change material is between 1 and 5 millimeters.

Thus, this thickness depends on the enthalpy of the layer—the higher the enthalpy, the lower the thickness can be—and on the arrangement of the device.

Generally, for the three layers of material, the objective is to find a compromise between a thickness which is as low as possible and a dissipation, and therefore an enthalpy or a conductivity, which is as high as possible.

Advantageously, since the three layers are first three layers, the device further comprises a fourth layer of heat-insulating material, situated between the three layers firstly and an end of the housing secondly.

Thus, this insulating layer prevents the temperature from increasing on the side of the housing opposite the heat source. In particular, this side is intended to be in contact with the ambient air, whose temperature may already be high and which must therefore be separated from the heat source and the heat dissipation system formed by the three layers.

Preferably, the conductivity of the layer of heat-insulating material is between 0.018 and 0.026 watts per meter-kelvin, this layer preferably comprising a heat-insulating sheet comprising a silica aerogel and nanofibers, the thickness of this layer preferably being between 0.1 and 1 millimeters.

In particular, suitable materials of thickness 500 micrometers providing sufficient thermal insulation for this end of the housing are available.

Advantageously, the device further comprises a gap pad situated between the heat source firstly and the layers of material secondly, the pad comprising a material whose thermal conductivity is preferably greater than or equal to 12.0 watts per meter-kelvin and whose thickness is preferably between 0.5 and 3 millimeters.

Thus, the gap pad collects the heat emitted by the heat source to transfer it to the first conducting layer. The heat source may be situated at different positions—a printed circuit will comprise, for example, several electronic elements which emit heat—or be situated at different levels, for example at different heights on the printed circuit. The gap pad, thanks to its housings formed during its assembly to the heat sources and adapted to the levels of the various heat sources, both maintains the heat source(s) in position and efficiently transfers the heat emitted to the adjacent layers of material.

Preferably, the heat source is at least a printed circuit component.

The flow and storage of electric charges generates heat in the circuit components which are for example resistors or more complex components forming processors or for example ASICs (Application-Specific Integrated Circuits). The printed circuit can be used to control or supply any electronic component of the device, such as a remote connection member, a screen, a communication terminal, any interface or a remotely-controlled or preprogrammed member performing a task on the device, such as a wave transmission module.

Advantageously, the heat source is a wave transmission module adapted to transmit waves of frequency between 3 and 120 gigahertz and surface power density greater than or equal to 0.5 milliwatts per square centimeter.

Thus, the device is used to transmit millimeter waves to the skin of a human subject, such as a wrist or ankle if the device is a bracelet. The electronics used to transmit these waves generate a large quantity of heat which must be dissipated. In particular, it may be necessary, for comfort and regulatory reasons, to limit the temperature of the device in contact with the skin to 43° C., especially if contact with the heat source exceeds ten minutes. Thus, in an embodiment of a connected device comprising such a module, the thermal power to be dissipated is approximately 1.25 watts for thirty minutes' operation. The device according to the present disclosure is designed in particular to dissipate this power for this duration, the various layers being part of the device dissipation management system.

Preferably, the device as described above is intended for use in the treatment of pain.

It is known, in fact, that the millimeter wave therapy (i.e. waves of frequency less than 300 gigahertz) reduces pain (see the publication by Usichenko T l, Edinge H, Gizkho W, Lehmann C, Wend M, Feyerherd F: “Low-intensity electromagnetic millimeter waves for pain therapy. Evid Based Complement Alternat Med”). It was thus demonstrated that exposing an area of the human body to millimeter electromagnetic waves led to the release of endogenous opioids (see the publication by Rojavin M A, Ziskin M C: “Electromagnetic Millimeter waves increase the duration of anaesthesia caused by ketamine and chloral hydrate in mice Int J Radiat Biol”), generating in the brain the synthesis of enkephalin, a natural peptide involved in the tolerance of pain. This technique can therefore be used to avoid the disadvantage of treatment by antalgics—undesirable side effects—or by neurostimulation—patient discomfort—and its operation is understood. Transmitting millimeter waves of surface power density of at least 0.5 milliwatts per square centimeter (and preferably above), over a small area of the human wrist, requires electronics which necessarily generate heat, which may correspond, as indicated above, to nearly 1.25 watts of thermal power emitted for thirty minutes. In particular, in this type of device, converting the direct current from a battery into electromagnetic radiation is very inefficient, generating the heat losses mentioned. In addition, better efficiency is achieved at low temperature. It is therefore important, both as regards the battery autonomy and to respect the patient's heating limits, to optimize the heat dissipation management. This is therefore achieved using the characteristics of the device dissipation management system described above.

Advantageously, the housing further comprises a temperature sensor adapted to stop the emission of heat from the heat source if the device temperature exceeds a predetermined threshold, for example if the temperature of the heat source exceeds 55° C.

Thus, this hardware safety device, independent of any software which would be implemented in the device, can switch off all power if this temperature of 55° C. is reached. Obviously, another temperature could be predetermined, but like all hardware safety devices, it is intended to be frozen from the device design stage and not to be modified, at any rate easily, by a user. It therefore acts as a last resort safety device if the software safety devices, which should have already stopped the heat source, have failed to operate as initially required.

Preferably, the device further includes an electronic medium comprising a stored computer program, the program comprising code instructions adapted to activate and/or deactivate emission of heat from the heat source depending on the device temperature, for example adapted to deactivate emission if the temperature is greater than 43° C. and activate emission if the temperature is less than 41° C.

Thus, one or more software safety devices are concerned. The first consists in pausing the system generating the heat source if the temperature of the heat source on the subject's skin exceeds 43° C. When the temperature has dropped below 41° C., the system can be restarted. A second software safety device can switch off all power if the temperature exceeds 50° C. This second safety device can be designed so that, once triggered, the device can only be reactivated by a predetermined person, for example a person from an after-sales service.

### BRIEF DESCRIPTION OF THE FIGURES

We will now describe one embodiment of the invention, as an example referring to the attached drawings in which:

FIG. 1 is a perspective view of a bracelet according to a first embodiment of the device and partially transparent;

FIG. 2 is a partially exploded view of the bracelet of FIG. 1;

FIG. 3 is a side and cross-sectional view of the bracelet of FIG. 1;

FIG. 4 is a side view with no transparency of the bracelet of FIG. 1;

FIG. 5 is a perspective view of a strand and of some layers of the bracelet of FIG. 1;

FIG. 6 is a similar exploded view of the same strand and of an additional layer;

FIG. 7 is a diagram of an arm model aimed at reproducing the heating conditions of the bracelet of FIG. 1;

FIG. 8 is a graph illustrating temperature measurements on the model of FIG. 7 and on the bracelet of FIG. 1; and

FIG. 9 is a graph illustrating other temperature measurements on the same elements.

### DETAILED DESCRIPTION

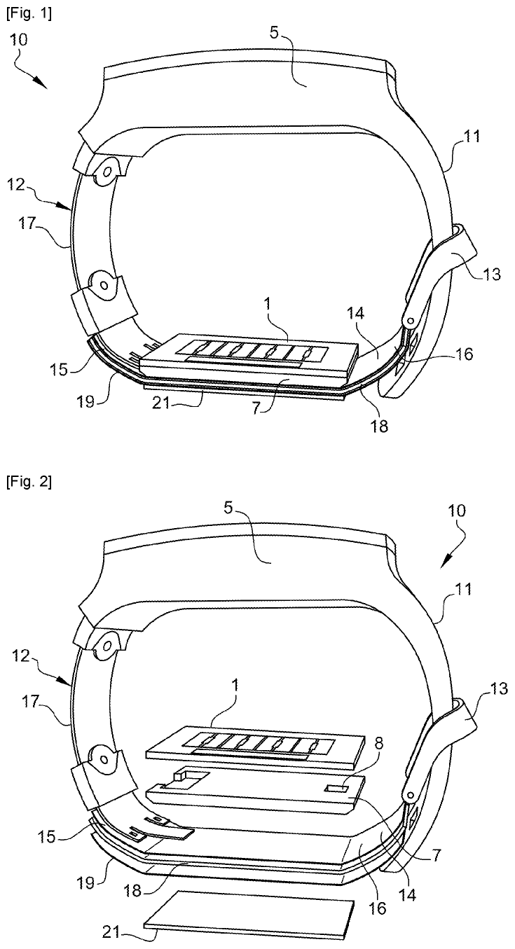

A bracelet 10 according to one embodiment of the invention is illustrated on FIGS. 1 to 4. This bracelet is intended to treat pain in a patient wearing the bracelet. By transmitting millimeter waves, this bracelet can also be used to treat stress or simply to generate a feeling of well-being in this patient.

The bracelet 10 transmits waves to the surface of the skin, in particular in the area marked Z on FIG. 4 and which corresponds to the lower part of the patient's wrist 6, in this case cut transversally. Fastened like a watch around the wrist 6, the bracelet 10 can thus be worn without any particular disadvantage by the patient and treat the patient without discomfort. The area Z corresponds to a portion of the human body which is particularly innervated. The study conducted by Radzievsky A A, Rojavin M A, Cowan A, Alekseev S I, Ziskin M C. Hypoalgesic effect of millimeter waves in mice: Dependence on the site of exposure. Life sciences. 2000; 66(21):2101-11” demonstrated the advantageous therapeutic effect of transmitting millimeter waves in such areas. It is therefore particularly interesting to wear the bracelet 10 to transmit waves in this area Z.

We will now describe the structure of the bracelet 10.

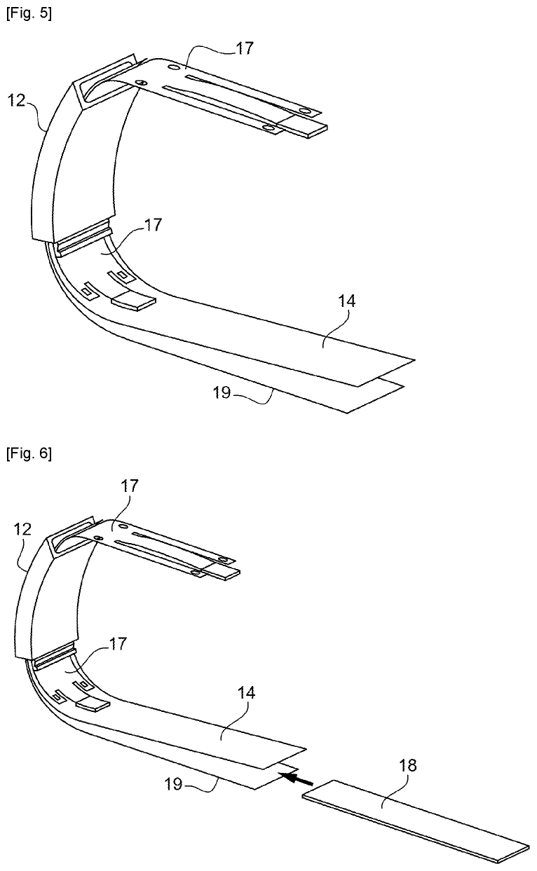

With reference to FIGS. 4 and 5, the bracelet 10 has a rigid housing 2 intended to be pressed against the lower part Z of the human wrist 6 when the bracelet 10 is closed around the wrist 6. The term rigid is intended to mean that the surfaces of the housing cannot easily be deformed by the human hand, like a watch housing for example. This rigid housing 2 is provided with a polycarbonate rigid internal wall 3 intended to be pressed against the lower part Z of the human wrist. The polycarbonate of this wall 3 allows the waves transmitted by the wave transmission module 1 described below to pass through. Alternatively, another material transparent to millimeter waves, such as silicone, can be used. The housing 2 is also provided, on the front, with a plastic rigid external wall 4 intended to be in contact with the ambient air. This housing 2 comprises elements which will be described below.

The bracelet 10 is also provided with two strands 11 and 12 extending respectively on each side of the housing 2 and from this housing. These two strands 11 and 12 comprise silicone and form flexible sections of the bracelet, adapting to the shape of the patient's wrist 6 like the strands of a traditional watch. The strand 11 can itself be subdivided into two sub-strands arranged to form a loop 13 in order to close and open the bracelet. This arrangement to close the loop 13 will not be detailed in this application and may be of any type. The two strands 11 and 12 extend from the housing 2 towards another rigid housing 5, which is intended to be placed on the upper part of the wrist 6, as is generally the case for the housing (with the dial) of a watch for example.

This housing 5 therefore extends opposite the housing 2, each one being pressed against two opposite parts of the patient's wrist, these two rigid housings 2 and 5 therefore being connected by flexible strands 11 and 12. This housing 5 comprises an interface allowing the patient or a doctor to configure the start of wave transmission. It also contains a battery and possibly other electronic elements which will not be described in detail. The electronics of this housing 5 are used to control and supply the wave transmission module 1 described below.

We will now describe the wave transmission and its consequences on the heat emitted.

To send waves to the surface of the skin, the rigid housing 2 contains a wave transmission module 1 placed against the internal wall 3 of the housing 2 which allows these waves to pass through. For the wave transmission to have a medical effect, the surface power density of the waves transmitted must be at least 0.5 milliwatts per square centimeter of irradiated skin. For the treatment to be effective, the frequency of these waves must be less than 300 gigahertz. We speak of “millimeter” waves. Concerning this subject, the study by Radzievsky A A, Gordiienko O V, Alekseev S, Szabo I, Cowan A, Ziskin M C: “Electromagnetic millimeter waves for pain therapy. Evid Based Complement Alternat Mede tends to demonstrate that the optimum effect of a millimeter wave treatment is obtained with a frequency of approximately 61.25 GHz and a surface power density of approximately 13 mW/cm². This module, illustrated on FIGS. 1 to 3, is provided with a silicon integrated circuit in a BGA (Ball Grid Array) type package comprising bumps, the circuit being soldered to a PTFE (polytetrafluoroethylene) HF substrate RO3003. This printed circuit, comprising arrangements between ASICs and surface antennas as well as a power supply circuit, is used to transmit waves to the skin, with the required surface density and frequency, for the required duration. In this case, the area of this module 1 is 20*37 millimeter, and the area irradiated by the waves is 2.5 square centimeters of skin. Obviously, the device is not limited to a particular module 1. For example, the dimensions and materials, as well as the electronic components, could be different.

The wave treatment is not continuous. For example, it may last a few minutes, be stopped, then start again. In one embodiment of a wave transmission treatment, the waves may be transmitted for thirty minutes continuously and so as to generate a radiated power absorbed by the skin of approximately 35 milliwatts. For such an absorbed power, the module 1 must be supplied with 1.25 watts. However, the energy conversion efficiency between the current received and the electromagnetic radiation (conversion carried out by the ASICs of module 1) is low. The heat losses are therefore high. In addition, a sharp temperature increase may be observed.

This temperature increase has several disadvantages. Firstly, the lower the temperature of the module 1, the higher the efficiency. The temperature of the module 1 must therefore be lowered. Secondly, the heat is transmitted to the skin, in particular the area Z of the patient's wrist 6. However, the temperature increase must not be uncomfortable for the patient. The patient's sensation may depend on numerous factors—his/her activity at the time, the size of the heating area, the rate of temperature increase—but it is generally considered that a maximum acceptable temperature is 43° C. This is in fact also the maximum temperature set by standard EN 60601-1, which stipulates that, for a contact duration of more than ten minutes between a medical device and a skin, the temperature of the part applied on the skin must not exceed 43° C. For all these reasons, the device provides for a heat dissipation management system within the bracelet 10 designed to dissipate 1.25 watts of thermal power for thirty minutes so that the highest temperature at a point of the patient's skin does not exceed 43° C.

We will now describe the structure of the heat dissipation management system contained within the bracelet 10.

Firstly, the bracelet 10 uses the patient's skin as a heat dissipation vector. Simulations have in fact demonstrated that, despite a small contact area, 25% of the heat is absorbed by the skin. This can be explained in particular by the high perfusion of the skin on the inside of the wrist 6. It is therefore relevant to optimize the area of the skin in contact with the heat source. This can be achieved firstly by pressing as much as possible the lower part of the wrist 6 against the wave transmission module 1, and secondly by dissipating the heat along the bracelet 10 and by pressing this bracelet as much as possible along the skin. This is partly achieved using the flexible strands 11 and 12, which adapt to the shape and dimensions of the patient's wrist 6. However, the heat must first be dissipated inside the bracelet so that it can then be absorbed by a greater area of skin.

This is why in particular the bracelet 10 comprises several layers of material intended to dissipate the heat emitted by the module 1 along the strand 12. This dissipation as such also reduces the temperature increase by spreading the heat as far away as possible from the module 1. We will now describe this stack of layers, which dissipates the heat emitted along the strand 12.

First, note that the strand 12 contains a flex 17, illustrated on FIGS. 5 and 6, used to connect the electronics of the housing 5 to those of the housing 2, in particular to those forming the wave transmission module 1.

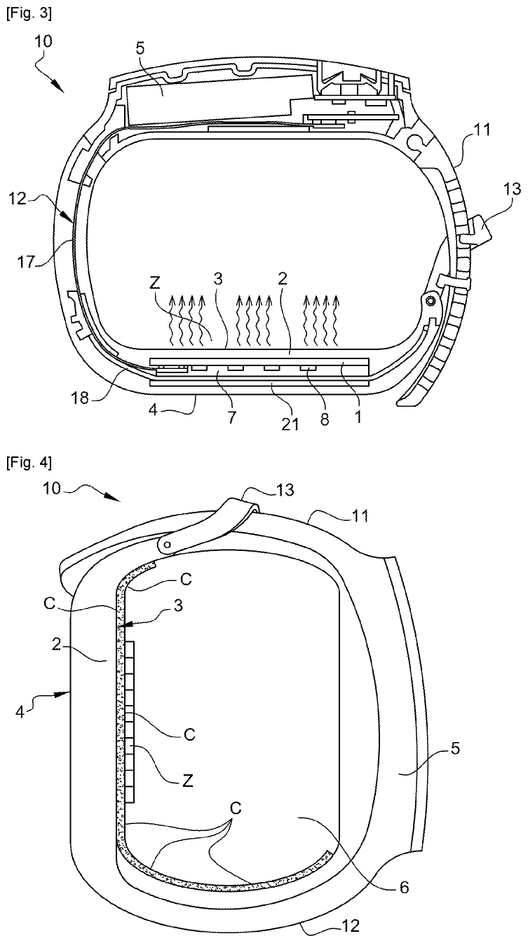

With reference to FIGS. 1 and 2, the housing 2 comprises a gap pad 7 of generally rectangular shape, used to house each ASIC of the module 1 in cavities formed during assembly—the gap pad being soft and deformable—for example the cavity 8 illustrated on FIGS. 2 and 3. The pad 7 thus acts as a housing for the components of the printed circuit of module 1. In particular, it is used to optimize the transfer of the heat emitted by these components to the next layer 14, described below. These components have in fact different sizes, different positions, such that, with its cavities placed at the various levels, the gap pad 7 can uniformly cover the entire circuit and transfer the heat emitted by the components, no matter where they are. In this case, the commercial reference of the illustrated pad 7, manufactured by T-global Technology, is “TGX-150-150 2.0-0”. Its area is adapted to the dimensions of the module 1 and it is 2 millimeters thick. The thermal conductivity of this pad 7 is 12 watts per meter-kelvin.

Other gap pads could be suitable. The objective is to find a compromise between the highest possible thermal conductivity and the cost of the pad. As regards the dimensions, the objective is to ensure good contact with the heat sources of the circuit, while minimizing the thickness. The pad thickness must therefore be at least equivalent to that of the highest component of the circuit, with a margin for example of 20 to 30%, while being as thin as possible.

With reference to FIG. 5, under the pad 7, the housing 2 is provided with a first layer 14 of conducting material, in this case a Pyrolitic Graphite Sheet (PGS) manufactured by Panasonic, reference EYGA091205PM, which has a thermal conductivity of 1300 watts per meter-kelvin, extends over a rectangular area of 115*20 millimeters and is 50 micrometers thick. This layer evacuates the heat from the module 1 transmitted by the pad 7 to the outside of the housing 2. This layer 14 extends from the housing 2 towards and into the strand 12 and towards the loop 13 of each side of the housing 2 and to the outside of this housing, within two curved portions 15 and 16. As illustrated on FIGS. 1 and 2, this layer 14 is attached by its portion 15 firstly to the flex 17 of the strand 12, and secondly via its portion 16, towards the loop 13. While the strand 11 is cut into two due to the closure loop 13, this is not the case for the strand 12. Thus, since the portion 15 of the layer 14 is bonded to the flex 17, the heat emitted by the module 1, transferred via the pad 7 to the layer 14, extends along the entire length of the layer 14 which has a high conducting power, then into the flex 17 along the strand 12. We therefore take advantage of the circumference of the bracelet 10, in particular of one of its flexible sections, in this case the strand 12, to dissipate as much as possible the heat emitted by the module 1. This dissipation is illustrated schematically on FIG. 4 where the heat is represented in an area C distributed on the housing 4, but also along the entire length of the strand 12 and towards the loop 13. This flow of heat, due to the high thermal conductivity of the layer 14, reduces the temperature increase of the module 1 and also transfers the heat over a larger area of skin, so that the temperature increase felt by the patient is reduced in two different ways.

Alternatively, the conducting layer 14 may have other dimensions. It may also be made of a different material. For example, it may include copper, which has a thermal conductivity of approximately 350 watts per meter-kelvin. However, the higher the conductivity, the more effective the dissipation and the lower the need for a thick layer, which saves space and makes the bracelet more flexible. A compromise must be found between cost, the highest possible conductivity and the lowest possible thickness. Generally, a reasonable thickness ranges from 0.01 to 1 millimeter so that the strand 12 remains flexible.

With reference to FIGS. 1 to 3, the housing is provided with a second layer 18, this time including a phase change material, of surface dimensions similar to those of the layer 14 and thickness of 0.740 millimeters, positioned next to the conducting layer 14. The material used in this case, manufactured by Panasonic, reference “TSS EYGP0309RJAA”, has a phase change enthalpy of approximately 100 joules per gram. This layer of phase change material is intended to reproduce, for the temperature, the behavior of a transient state to prevent the temperature from increasing too suddenly. Thus, to avoid reaching the thermal equilibrium too rapidly, inertia is added to the system, via this layer of phase change material, to obtain a transient state since the inertia thus added prevents an equilibrium from being reached in a time less than the treatment duration. This layer 18 of phase change material therefore acts as a gap pad storing the heat dissipated during the temperature increase and which is transferred to the pad by the layer 14, to restore it later. In concrete terms, it can absorb approximately 450 joules, i.e. 20% of the 2250 joules (1.25 watts for 30 minutes) of heat emitted during the wave transmission. This layer of phase change material 18 is sandwiched between the conducting layer 14 and another conducting layer 19.

Obviously, the layer of phase change material 18 could be made of another material with similar properties. This type of material generally includes microparticles of paraffin or other materials with similar properties, with in particular a melting point, and therefore phase change temperature, of approximately 35 to 40° C. Its dimensions, in particular its thickness, may also be different. Generally, the volume and therefore the possible energy storage capacity of this layer 18 of phase change material must be sufficiently large. In addition, it must be easy to access this phase change material; if it is too thick, the heat must first diffuse through it before the material changes phase. This is why in particular this layer of phase change material is surrounded by two conducting layers, the layer 14 and the layer 19. They optimize the diffusion of heat within the layer of phase change material. By seeking to find a reasonable compromise between thickness and ability to change phase rapidly, a reasonable thickness of the layer of phase change material should be between 1 and 5 millimeters.

The third layer 19 is a heat-conducting layer similar to the layer 14 in terms of its dimensions and identical in terms of its material of high conducting power. It is positioned against the layer 18 of phase change material so that the two similar conducting layers 14 and 19 surround the layer 18 of phase change material. This layer 19 is thus also attached, at the flex 17, to the layer 18 of phase change material.

Alternatively, it may be made of a material different from that of the layer 14. Once again, the objective is to find a compromise between a reasonable thickness, the highest possible conductivity and the lowest possible costs. For example, this layer 18 could be made of copper while the layer 14 would still be made of graphite.

As illustrated in particular on FIG. 2, the last layer 21 is limited, in terms of dimensions, to the length of the rigid housing 2. Thus, unlike the layers 14, 18 and 19, the layer 21 does not extend outside the rigid housing 2. This layer 21, positioned against the outer rigid wall 4 of the housing 2, opposite the wall 3 from where the waves are transmitted, is thermally insulating. It can therefore limit the temperature increase on the housing 2 on the side 4 opposite the wall 3 from where the waves are transmitted. It is therefore the layer inside the housing 2 closest to the ambient air so that, if the temperature of the ambient air increases, the hot spot corresponding to the presence of components dissipating the heat is masked from the outside of the housing 2. Thus, the thermal resistance on the surface of the housing 2 on which this insulating layer 21 is placed, is increased, forcing the heat to propagate further along the bracelet or towards the skin. Consequently, the temperature does not exceed the maximum permissible value at the point closest to the components.

This layer is made of a material manufactured by Panasonic, reference “NASBIS EYGY0912QN4S” and is 500 micrometers thick. Obviously, this material and its thickness could be different. The objective is to find a compromise between a sufficiently low thickness and a sufficiently high ability to thermally insulate the housing.

We will now summarize the structure of the housing 2.

Structurally, the housing 2 therefore contains a stack includes, starting from the wall 3 next to the human wrist 6, the wave transmission module 1 including the heat source electronics, the gap pad 7, then a stack of three layers 14, 18 and 19 projecting out of the housing 2 to extend into the strand 12 and towards the loop 13, and in particular attached to the electronic flex 17, the two conducting layers 14 and 19 surrounding the layer 18 of phase change material. Lastly, an insulating layer 21 not projecting out of the housing 2 is placed under the layer 19.

The heat emitted by the module 1 is therefore dissipated in the layer 14, which takes advantage of the flexible portions of the bracelet, in particular of the strand 12, to extend the heat dissipation area as much as possible. Thus, the heat leaves the area of the module 1 and spreads partly around the human wrist. This prevents the temperature increase from being concentrated at a single point. The heat is also transmitted to the layer 18 of phase change material, thereby absorbing some of the thermal power, to restore it progressively later, when the wave transmission has stopped. This layer 18, which also takes advantage of the flexible portions 11 and 12 of the bracelet to extend its absorption volume and to recover the heat dissipated by the layer 14, therefore captures some of the heat. Lastly, the remaining heat is transmitted to the layer 19 to be dissipated over the largest possible area, in other words once again by extending over the flexible portions 11 and 12 of the bracelet. The insulating layer 21 masks the hot spot of the housing from the outside of the housing.

The housing 2 may including other components, for example one or more temperature sensors situated at different positions to monitor the temperature increase of the patient's skin and/or of the transmission module 1.

We will now describe the safety mechanisms designed to prevent an excessive temperature increase.

The first are software safety mechanisms. The housing 2 or the rigid housing 5 includes, in fact, in a storage medium, a computer program designed to control the wave transmission. Alternatively or in addition, this program may be stored remotely. In all cases, a program, controlled or configured by a user, automates the wave transmission, according to a prescribed medical treatment or a command by a user. This program therefore includes two safety mechanisms: the first interrupts the wave transmission if the skin temperature, determined by a sensor provided for this purpose and situated on the bracelet in the rigid housing 2, exceeds 43° C. When the measured temperature drops below 41° C., the wave transmission can resume. This therefore ensures that the skin temperature does not exceed 43° C., in compliance with the applicable standard. A second software safety mechanism, in other words included in the program controlling the wave transmission, stops the wave transmission permanently if the temperature should exceed 50° C. This safety mechanism is necessary if the previous safety mechanism should malfunction or in case of sudden, high temperature increase. With this second safety mechanism, the transmission can only be reactivated by an approved person, for example from an after-sales service.

The third is a hardware safety mechanism. This is the last emergency safety mechanism, if the computer program should fail to operate, for example if it is deleted or modified maliciously of inadvertently. If the temperature sensor in contact with the skin detects a temperature greater than or equal to 55° C., the wave transmission module is switched off directly by an electronic circuit provided for this purpose, that those skilled in the art know how to determine. It may for example use a component manufactured by Texas Instruments reference “TMP302ADRL” which switches off the power supply of the ASICs of the module 1 if the temperature exceeds a predetermined value, in this case 55° C.

These safety mechanisms are thus intended to eliminate any risk of uncontrolled temperature increase.

We will now describe the thermal bench which was used to approve the bracelet and demonstrate that the temperature did not rise above that provided for in standard EN-60601-1, thus validating the efficiency of the heat dissipation management system described previously.

Put briefly, standard EN-60601-1, paragraph 11.1.1, states that the temperature of a part of a medical device in contact with the skin cannot exceed 43° C. at the most unfavorable position for more than 10 minutes. The bracelet aims to comply with this standard, since the wave transmission may last for more than 10 minutes, in particular 30 minutes. Paragraph 11.1.3 of the standard defines how the device must be tested to measure the thermal heating.

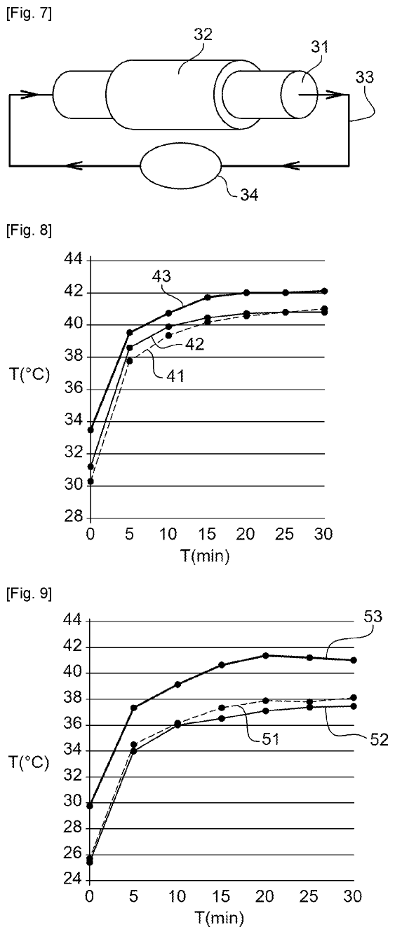

FIG. 7 illustrates the model used to reproduce the thermal properties of the human skin to test the bracelet. It therefore includes a duct 31 reproducing an arm, a 2% agarose gel 32 simulating the skin of the arm forming a sleeve placed on the duct, the bracelet 10 being placed on the sleeve. This gel is kept at a temperature similar to that of the human skin by the flow of a fluid 33 inside the duct. This fluid also reproduces the blood circulation, which evacuates some of the heat produced by the heating of the bracelet 10. The member 34 is a device used to keep the fluid 33 at a required temperature, the temperature being that used to obtain for the gel 32 a temperature similar to that of the skin.

Firstly, the temperature measurements on ten human participants and on the bracelet 10 were compared, to calibrate the temperature and the flow rate of the fluid 33, by varying them. Once the temperatures measured on the model were the same as those measured on the human participants, the corresponding fluid temperature and the fluid flow rate were determined.

It was then possible to take measurements on the model reproducing the conditions under which a bracelet is worn on a human being. The results are illustrated on FIGS. 8 and 9. FIG. 8 illustrates the temperatures of the skin in contact with the housing 2, with three curves: the curve 41 is the measurement of the average temperature on 10 human participants, for 30 minutes transmission, with an ambient temperature of 25° C. The curve 42 is the measurement taken on the model of FIG. 7, still at 25° C. We see that these curves are very close, the model even tending to overestimate the skin temperature. By comparing the curves 41 and 42, it was possible to validate the relevance of the model, which can easily be reproduced in laboratory. Lastly, the curve 43 represents the temperature taken on the model, with an ambient temperature of 30° C. This temperature of 30° C. corresponds to the ambient temperature above which the user manual will recommend that the bracelet 10 should not be used. We see that none of the curves exceeds the temperature of 43° C. At 30° C., the model reaches a maximum temperature of 42° C. and stabilizes at this temperature after 20 minutes. At 25° C., the two curves 41 and 42 show that the maximum temperature reached on the skin is less than 41° C. FIG. 9 illustrates three curves 51, 52 and 53 corresponding to the temperature taken on the side of the wall 4 of the housing, at 25° C. on a human being (average over 10 participants) for the curve 51, on the model of FIG. 7 for the curve 52 and at 30° C. for the same model on the curve 53. We see that no temperature exceeds 43° C., or even 42° C.

These tests were used to manufacture a test model adapted to the requirements of the standard, and to demonstrate that, in the worst case—thermal power of 1.25 watts for 30 minutes—at the most critical position—between the module and the skin—the temperature does not exceed 43° C. This temperature limitation is achieved thanks to the heat dissipation management system described above.

The invention is not limited to the embodiments described and other embodiments will be clearly apparent to those skilled in the art. In particular, the heat dissipation management system can be used in another type of bracelet, for example in a connected watch equipped with one or more applications and which would not be intended to transmit waves. For example, the bracelet may have only one rigid housing. In addition, this system could be adapted to another type of device such as a smartphone where it would manage the heat dissipation for example of the smartphone motherboard. This could involve adapting the dimensions of the various layers, in particular of the conducting layers, to take full advantage of the area available to dissipate heat.

Lastly, the housing 2 does not have to be rigid. It could, for example, be a “semi-rigid” housing or even a flexible housing. The embodiments described previously also apply for a non-rigid or non strictly-rigid housing, since the heat dissipation strategy does not depend on the flexibility of the housing.

1-19. (canceled)

20. A device, comprising:
at least one housing having at least one heat source, and
at least one strand forming a more flexible part than the housing,
the strand comprising two layers of heat-conducting material and a layer of phase change material situated between the two layers of heat-conducting material.

21. The device of claim 20, wherein the device is a bracelet.

22. The device of claim 20, wherein the housing is rigid.

23. The device of claim 20, wherein a thermal conductivity value of at least one of the heat-conducting layers is greater than or equal to 350 watts per meter-kelvin.

24. The device of claim 20, wherein a thickness of at least one of the heat-conducting layers is between 0.01 and 1 millimeter.

25. The device of claim 20, wherein a phase change enthalpy of the layer of phase change material is approximately 100 joules per gram.

26. The device of claim 20, wherein the two layers of heat-conducting material are bonded to two opposite surfaces of the layer of phase change material.

27. The device of claim 20, wherein at least one of the three layers extends into the housing.

28. The device of claim 20, wherein an electronic connection flex extends from the housing and into the strand, at least one of the layers of heat-conducting material being attached to the flex in the strand.

29. The device of claim 20, wherein at least one of the layers of heat-conducting material comprises a sheet of pyrolytic graphite having a thickness between 10 and 100 micrometers.

30. The device of claim 20, wherein a thickness of the layer of phase change material is between 1 and 5 millimeters.

31. The device of claim 20, wherein the device further comprises a second layer of heat-insulating material, situated between the two layers of heat-conducting material and the layer of phase change material and an end of the housing.

32. The device of claim 31, wherein the conductivity of the second layer of heat-insulating material is between 0.018 and 0.026 watts per metre-kelvin, the second layer comprising a heat-insulating sheet comprising a silica aerogel and nanofibers, a thickness of the second layer being between 0.1 and 1 millimetres.

33. The device of claim 20, wherein the device further comprises a gap pad situated between the heat source firstly and the layers of material secondly, the pad comprising a material whose thermal conductivity is greater than or equal to 12.0 watts per metre-kelvin and whose thickness is between 0.5 and 3 millimetres.

34. The device of claim 20, wherein the heat source is at least a printed circuit component.

35. The device of claim 20, wherein the heat source is a wave transmission module adapted to transmit waves of frequency between 3 and 120 gigahertz and surface power density greater than or equal to 0.5 milliwatts per square centimetre.

36. The device of claim 20, for use in treatment of pain.

37. The device of claim 20, wherein the housing further comprises a temperature sensor adapted to stop an emission of heat from the heat source if the device temperature exceeds a predetermined threshold, for example if the temperature of the heat source exceeds 55° C.

38. The device of claim 20, further comprising an electronic medium comprising a stored computer program, the program comprising code instructions adapted to activate and/or deactivate emission of heat from the heat source depending on a device temperature.
